(19) United States
(12) Patent Application Publication
(10) Pub. No.: US 2020/0315605 A1
(43) Pub. Date: Oct. 8, 2020

(54) BONE ANCHOR HAVING DEFORMABLE MEMBERS

(71) Applicant: Acumed LLC, Hillsboro, OR (US)

(72) Inventor: John Krumme, Bainbridge Island, WA (US)

(21) Appl. No.: 16/840,112

(22) Filed: Apr. 3, 2020

Related U.S. Application Data
(60) Provisional application No. 62/829,908, filed on Apr. 5, 2019.

(51) Int. Cl. A61B 17/04; A61F 2/08

(52) U.S. Cl. CPC A61B 17/0401; A61B 2017/00867; A61B 2017/0409; A61F 2/0811

(57) ABSTRACT

The provided system includes an inserter, a bone anchor, and one or more sutures. The inserter includes a tube and a plunger within the tube's interior. The bone anchor is structured with multiple deformable members such that it can be transitioned from a compact configuration to an expanded configuration. When inserting the bone anchor, the bone anchor may initially be in a compact configuration within the inserter tube. The inserter tube may be inserted into a bone hole. Then, while the plunger is maintained in its positioned, the tube may be retracted so that the bone anchor is deployed from within the tube. The bone anchor may then be transitioned to an expanded configuration. In some instances, suture tension transitions the bone anchor. In other instances, a pre-set shape of a shape memory material transitions the bone anchor.

### PRIORITY CLAIM

The present application claims priority to and the benefit of U.S. Provisional Application 62/829,908, filed Apr. 5, 2019, the entirety of which is herein incorporated by reference.

### BACKGROUND

Bone anchors are used extensively in open and minimally invasive surgery to reattach tissue to bone. The bone anchor is secured to the bone and one or more sutures attached to the bone anchor are used to secure the tissue to the bone. The tissue can be soft tissue anywhere in the body, for example, a torn rotator cuff in a shoulder or a torn ligament. One example of a bone anchor is disclosed in U.S. Pat. Nos. 8,721,650 and 9,539,001. The entire disclosure of each of those patents is incorporated herein by reference for all purposes.

Typically, in a first step to reattach tissue to bone, a hole is drilled into the bone under arthroscopic visualization. The bone anchor may be inserted into the hole and may be configured to lock itself within the hole in the bone upon deployment therein. Several means for securing the bone anchor within the hole of a bone are known in the art. Once the bone anchor is secured within the hole in the bone, one or both ends of suture attached to the bone anchor may be tensioned to approximate the positioning of the tissue with respect to the bone. Once the tissue is positioned as desired, the suture may be locked in place to maintain the tension in the suture. The free end or ends of the suture may be clipped under arthroscopic visualization to complete the procedure.

The bone hole size that is drilled when inserting the bone anchor corresponds to a patient's recovery time. Accordingly, a smaller bone hole size is desired to reduce recovery times for patients. A bone anchor, however, must have sufficient pull-out strength so that it stays in place and allows tissue to properly reattach to bone. Therefore, a bone anchor must be small enough to be inserted through a bone hole and also have reliable pull-out strength such that it is not pulled back through the bone hole. Further, bone hole sizes may vary depending on the location of the procedure in the body. It is therefore desired that a bone anchor may be configured in different sizes.

Accordingly, there is a need for a bone anchor that provides the above features.

### SUMMARY

The present disclosure provides new and innovative systems and methods for bone anchor insertion. In an example, a system includes an inserter, a plunger, a bone anchor, and at least one suture. The inserter includes a tube. The plunger is to be depressed through an interior of the tube. The bone anchor includes an insertion end and a plunger end. The plunger end is connected to the insertion end by a plurality of deformable members. Each respective deformable member includes a first portion, a second portion, and a third portion, the third portion being between the first and second portions. The third portion causes the respective deformable member to deform. When the bone anchor is in a compact configuration, the respective first portions are substantially parallel to the respective second portions. When the bone anchor is in an expanded configuration, the respective first portions are at an angle to the respective second portions. The bone anchor is positioned within the interior of the tube when the bone anchor is in the compact configuration. Forcing the insertion end towards the plunger end of the bone anchor in the compact configuration causes the bone anchor to transition to the expanded configuration. The at least one suture is coupled to the insertion end of the bone anchor.

In another example, a system includes an inserter, a plunger, a bone anchor, and at least one suture. The inserter includes a tube. The plunger is to be depressed through an interior of the tube. The bone anchor includes an insertion end and a plunger end. The plunger end is connected to the insertion end by a plurality of deformable members. Each respective deformable member includes a first portion and a second portion. The bone anchor is constructed of a shape memory material. When the bone anchor is in a compact configuration, the respective first portions are substantially parallel to the respective second portions. When the bone anchor is in an expanded configuration, the respective first portions are at an angle to the respective second portions. The bone anchor is positioned within the interior of the tube when the bone anchor is in the compact configuration. Upon the bone anchor exiting the interior of the tube, the bone anchor transitions from the compact configuration to the expanded configuration. The at least one suture is coupled to the insertion end of the bone anchor.

In another example still, a method includes inserting a bone anchor coupled to an inserter through a bone. The inserter includes a tube and a plunger that is to be depressed through an interior of the tube. The bone anchor includes an insertion end and a plunger end. The plunger end is connected to the insertion end by a plurality of deformable members. Each respective deformable member includes a first portion and a second portion. The bone anchor is initially in a compact configuration within the interior of the tube. The respective first portions are substantially parallel to the respective second portions when the bone anchor is in a compact configuration. The at least one suture includes a coupling end and a free end, and is coupled to the insertion end of the bone anchor at the coupling end. The tube is retracted while maintaining a positioning of the plunger, thereby causing the bone anchor to exit from the interior of the tube. The bone anchor is then transitioned from the compact configuration to an expanded configuration. When the bone anchor is in the expanded configuration, the respective first portions are at an angle to the respective second portions. The inserter is then removed from the bone.

### BRIEF DESCRIPTION OF THE DRAWINGS

FIG. 1A illustrates a cross-sectional view of an example bone anchor insertion system in a pre-deployment configuration, according to an aspect of the present disclosure.

FIG. 1B illustrates a side view of the example bone anchor insertion system of FIG. 1A in a post-deployment configuration, according to an aspect of the present disclosure.

FIG. 2 illustrates a perspective view of an example bone anchor having symmetric deformable members, according to an aspect of the present disclosure.

FIG. 3 illustrates a perspective view of an example bone anchor having symmetric deformable members, according to an aspect of the present disclosure.

FIG. 4 illustrates a cross-sectional view of a middle portion of an example bone anchor having deformable members, according to an aspect of the present disclosure.

FIG. 5 illustrates a perspective view of an example bone anchor having asymmetric deformable members, according to an aspect of the present disclosure.

FIG. 6 illustrates a side view of an example bone anchor insertion system, having a bone anchor with asymmetric deformable members, in a post-deployment configuration, according to an aspect of the present disclosure.

FIG. 7 illustrates an example method of inserting a bone anchor, according to an aspect of the present disclosure.

### DETAILED DESCRIPTION

The present disclosure provides new and innovative systems and methods for bone anchor insertion. The provided system includes an inserter, a bone anchor, and one or more sutures. The inserter includes a tube and a plunger within the tube's interior. The tube and the plunger may translate with respect to one another. The bone anchor is structured with multiple deformable members such that it can be transitioned from a compact configuration to an expanded configuration when the bone anchor's opposite ends are forced towards one another. When in a compact configuration, the deformable members are substantially straight, whereas in an expanded configuration the deformable members expand outward, forming an angle.

The deformable members are manufactured in the bone anchor by cutting slots in the bone anchor so that each of the deformable members are separated from one another by a slot. Each of the deformable members includes a zone of weakness that causes the deformable member to bend at the zone of weakness in particular when the bone anchor is transitioned to the expanded configuration. For instance, the zone of weakness may be narrower portion of material than the rest of the deformable member, which causes the deformable member to deform at the narrower portion. The bone anchor may be manufactured with zones of weakness at particular locations on the respective deformable members to obtain various features of the bone anchor.

When inserting the bone anchor, the bone anchor may initially be in a compact configuration within the inserter tube. The inserter tube may be inserted into a bone hole. Then, while the plunger is maintained in its positioned, the tube may be retracted so that the bone anchor is deployed from within the tube. The bone anchor may then be transitioned to an expanded configuration and secured with suture.

Accordingly, the bone anchor may be small and compact when it is inserted through the bone hole via the inserter, while also expanding at a desired location in the bone to provide suitable pull-out strength. The provided bone anchor insertion system therefore enables smaller bone holes without sacrificing pull-out strength. Additionally, the bone anchor may be manufactured with a variety of deformable member shapes, thus increasing the flexibility of the provided bone anchor as compared to typical bone anchors.

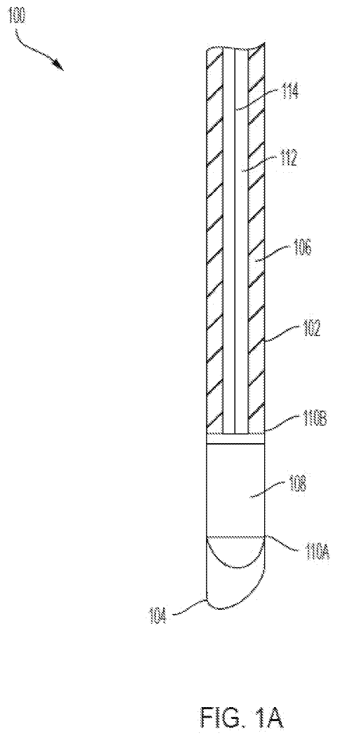

FIG. 1A illustrates a cross-sectional view of an example bone anchor insertion system 100 in a pre-deployment configuration. The example system 100 includes an inserter, a bone anchor 108, and a suture 114. In some instances, the example system 100 may include more than one suture 114. The inserter includes a tube 102 and a plunger 106 that is inserted within the tube 102. In various instances, the tube 102 is cylindrical. The tube 102 may include a handle, or may otherwise terminate, at one end. On its other end, the tube 102 may be shaped such that it can facilitate insertion of the tube 102 into the bone. For example, the tube may be inserted into the bone by tapping the tube 102 into place with a small mallet. In such instances, the tube 102 may include a pointed tip 104. The pointed tip 104 may be dull, but otherwise pointed, as illustrated in FIG. 1A. In some examples, the tube 102 may include markings on its exterior to indicate how far the tube 102 has been inserted into bone. The tube 102 may be constructed of a suitably rigid, biocompatible material. For instance, the tube 102 is suitably rigid to maintain the bone anchor 108 in a compact configuration when the bone anchor 108 is positioned within the tube 102, as will be described in more detail below.

The plunger 106 is structured such that the plunger 106 and the tube 102 can be translated back and forth with respect to one another. One end of the plunger 106 is structured to contact the bone anchor 108. The other end of the plunger 106 is structured such that pressure may be applied to it during deployment of the bone anchor 108. A surgeon may manually apply pressure directly to the plunger 106 or may remotely control pressure applied to the plunger 106. In some examples, pressure applied to the plunger 106 may hold the plunger 106 stable while the tube 102 is retracted to deploy the bone anchor 108 from the interior of the tube 102. In other examples, pressure may be applied to the plunger 106 while the tube 102 is held stable so that the plunger 106 translates and deploys the bone anchor 108 from the interior of the tube 102. The plunger 106 may have a shape that corresponds to the shape of the tube 102. For example, in instances in which the tube 102 is cylindrical, the plunger 106 may also be cylindrical. In such instances, the bone anchor 108 may be cylindrical as well. The plunger 106 may be hollow such that it includes an interior channel 112.

The bone anchor 108 is constructed of one or more implant-compatible materials, such as titanium, a nickel-titanium alloy, stainless steel, or a polymeric composition including a polymer such as PEEK, PMMA, or ultra-high molecular weight polyethylene. In aspects in which the bone anchor 108 is constructed of a polymer, the bone anchor 108 may be manufactured by, for example, injection molding. In some instances, the material of the bone anchor 108 may be selected based on a density of the bone for which it will be used. For example, titanium or stainless steel may be used for harder, denser bone, whereas a nickel-titanium alloy or a polymeric composition may be used for softer, less dense bone.

The bone anchor 108 includes an insertion end 110A and a plunger end 110B. The insertion end 110A may include a suture anchor tip 118 that is molded or shaped to retain one or more sutures 114. Stated differently, when one portion of the suture 114 is retained by the suture anchor tip 118, and the opposite portion of the suture 114 is pulled while the suture anchor tip 118 is held stationary, tension is created in the suture 114. For example, the suture anchor tip 118 may include a pin configured such that a slidable loop of suture 114 may slide over the pin to secure it to the pin. Suture 114 retained by the suture anchor tip 118 is positioned through the interior of the bone anchor 108 and through the interior channel 112 of the plunger 106 so that a surgeon may access the suture 114 from the other end of the tube 102 either manually or remotely via a robotic surgical instrument. In some instances, the suture 114 is wound onto a release mechanism at the other end of the tube 102.

The suture 114 may typically be a polyethylene woven or braided, high-strength suture. In other examples, however, the suture 114 may be other implant-compatible materials. The suture 114, in some aspects, can be secured to the suture anchor tip 118 such that the suture 114 is prevented from sliding. For example, the suture 114 may be knotted at the suture anchor tip 118. In another example, the suture 114 may be heat formed into a shape that retains the suture 114 within the bone anchor 108.

Additionally, the bone anchor 108 is configured such that it may be in a compact configuration (FIG. 1A) or an expanded configuration (FIG. 1B). Forcing the insertion end 110A towards the plunger end 110B transitions the bone anchor 108 from the compact configuration to the expanded configuration by expanding multiple deformable members outward with respect to a central axis of the bone anchor 108. This configuration enables the bone anchor 108 to be compact when inserted through a bone hole, but expanded when properly positioned within a patient to provide suitable pull-out strength. The provided bone anchor insertion system accordingly enables both small bone holes and a bone anchor with suitable pull-out strength.

As stated above, to enable the transition from the compact configuration to the expanded configuration, the bone anchor 108 includes multiple (e.g., 2, 3, 4, 5, 6, 7, 8 etc.) deformable members between the insertion end 110A and the plunger end 110B. A bone anchor 108 having deformable members can be manufactured, for example, by cutting (e.g., laser cutting) slots in the bone anchor. The material of the bone anchor 108 between each slot constitutes a deformable member. Each of the slots starts or terminates at a suitable distance from the insertion end 110A and the plunger end 110B to help ensure a durability of the bone anchor 108. For instance, too little material between a slot and an end of the bone anchor 108 may cause the material to rip at that location. A bone anchor 108 having deformable members can therefore be simply and economically manufactured.

FIG. 1B illustrates a side view of the example bone anchor insertion system 100 of FIG. 1A after the bone anchor 108 has been deployed from the inserter. In this example, the bone anchor 108 is in the expanded configuration and includes two deformable members that are expanded outwards. In typical examples, the bone anchor 108 may have a width (e.g., diameter) between 1 mm and 4 mm in the compact configuration, though this is not limiting. In various instances, the maximum width of the bone anchor 108 in the expanded configuration may be between two to four times the width of the bone anchor 108 in the compact configuration. In other instances, the maximum expanded configuration width may be less than two times or greater than four times the compact configuration width. In at least one example, the maximum expanded configuration width is three times the compact configuration width. The maximum width of the bone anchor in the expanded configuration depends at least in part on the length of the deformable members. For example, longer deformable members are able to expand farther outward when they are deformed. Additionally, a greater maximum width of the expandable configuration may help increase pull-out strength of the bone anchor 108 in some instances.

The first deformable member of the bone anchor 108 includes a zone of weakness 122 in between a first portion 116A and a second portion 116B. The second deformable member includes a zone of weakness 124 between the first portion 120A and a second portion 120B (i.e., the zone of weakness 124 may be a third portion distinct from the first portion and the second portion). When the bone anchor 108 is in a compact configuration (e.g., within the tube 102 in FIG. 1A), the first portion 116A is substantially parallel to the second portion 116B, and the first portion 120A is substantially parallel to the second portion 120B. Stated differently, the perimeter of the bone anchor 108 is substantially linear or straight.

When the bone anchor 108 is in an expanded configuration (e.g., FIG. 1B), the first portion 116A is at an angle to the second portion 116B, and the first portion 120A is at an angle to the second portion 120B. The respective angles may vary depending on, for example, the construction of the deformable members, the amount of force applied to force the insertion end 110A towards the plunger end 110B, the bone anchor 108 material, and other contributing factors. Stated differently, the bone anchor 108 may be adjusted through many different positions in the compact configuration by adjusting the distance between the insertion end 110A and the plunger end 110B.

In some instances, the bone anchor 108 may transition from the compact configuration to the expanded configuration due to tension in the suture 114. For example, the tube 102 may be retracted while the plunger 106 remains stable so that the bone anchor 108 is deployed from within the interior of the tube 102. The suture 114, which is retained by the suture anchor tip 118, may then be tensioned (e.g., by being pulled), thereby forcing the insertion end 110A of the bone anchor 108 towards the plunger end 110B because the plunger 106 prevents the bone anchor 108 from moving.

In other instances, the bone anchor 108 may be constructed of a shape memory material and may transition from the compact configuration to the expanded configuration as the result of a pre-set shape of the shape memory material. The shape memory material may be a superelastic nickel-titanium alloy, such as nitinol. For example, the bone anchor 108 having a pre-set shape in the expanded configuration may be compressed into the compact configuration and inserted into the tube 102 of the inserter, which maintains the bone anchor 108 in the compact configuration. When the bone anchor 108 is deployed, and is no longer constrained by the walls of the tube 102, the bone anchor 108 returns to its pre-set shape in the expanded configuration. In some instances, the distance between the insertion end 110A and the plunger end 110B of the shape-memory bone anchor 108 may be further adjusted using the suture 114 as described above. For instance, the pre-set shape may provide suitable pull-out strength in some instances, whereas in other instances, a surgeon may adjust the expanded shape-memory bone anchor 108 after inserting it to obtain a desired pull-out strength.

Additionally, as illustrated in FIG. 1B, the respective angles are located at the zone of weakness 122 and the zone of weakness 124. Stated differently, in some instances, the deformable members are configured such that they deform at their respective zones of weakness 122 and 124. In aspects in which the bone anchor 108 is constructed of a shape memory material with a pre-set shape, the deformable members do not necessarily have a zone of weakness as it is described herein. Instead, the shape memory material's properties may determine the location at which the deformable members deform. In some instances, a bone anchor 108 constructed of a shape memory material also includes deformable members having respective zones of weakness.

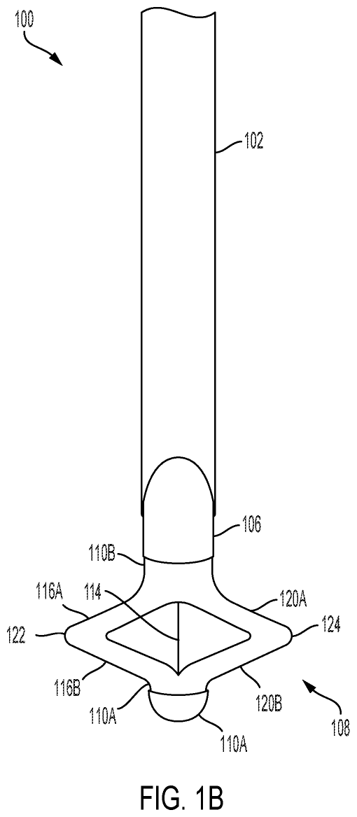

FIG. 2 illustrates a perspective view of an example bone anchor 200 having symmetric deformable members. The bone anchor 200 includes a plunger end 202 and an insertion end 204. Additionally, the bone anchor 200 includes multiple cut-out slots, such as the slots 206A and 206B. The slots 206A and 206B are an absence of material. Between the slot 206A and the slot 206B is a deformable member that includes a zone of weakness 212 between a first portion 208 and a second portion 210. The deformable member is symmetric in this example because the first portion 208 has an equal length with the second portion 210. Stated differently, the zone of weakness 212 is positioned in the center of the deformable member.

The zone of weakness 212 has a narrower width than the first portion 208 and the second portion 210, which causes the deformable member to deform at the zone of weakness 212, rather than at another location, when the bone anchor 200 is transitioned to the expanded configuration. In this example, the narrower width of the zone of weakness 212 is created by the design of the slots 206A and 206B that remove extra material adjacent to the zone of weakness 212 as compared to adjacent to the first and second portions 208 and 210. It should be appreciated that there may be multiple slots identical to the slots 206A and 206B around the perimeter of the anchor 200 that create multiple deformable members.

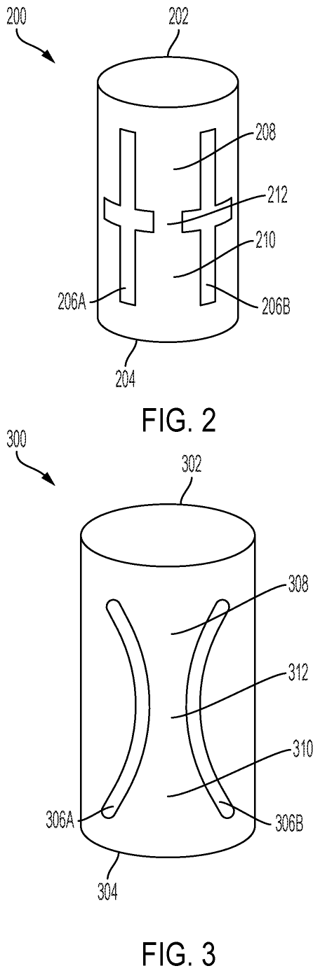

FIG. 3 illustrates a second example of a bone anchor having symmetric deformable members. The bone anchor 300 includes a plunger end 302 and an insertion end 304. Additionally, the bone anchor 300 includes multiple cut-out slots, such as the slots 306A and 306B. The slots 306A and 306B are an absence of material. Between the slot 306A and the slot 306B is a deformable member that includes a zone of weakness 312 between a first portion 308 and a second portion 310. The deformable member is symmetric in this example because the first portion 308 has an equal length with the second portion 310. Stated differently, the zone of weakness 312 is positioned in the center of the deformable member.

The zone of weakness 312 has a narrower width than the first portion 308 and the second portion 310, which causes the deformable member to deform at the zone of weakness 312, rather than at another location, when the bone anchor 300 is transitioned to the expanded configuration. In this example, however, the narrower width of the zone of weakness 312 is created by the tapered design of the slots 306A and 306. Each of the slots 306A and 306B converge towards one another symmetrically to create a deformable member with a tapered or hourglass shape. It should be appreciated that there may be multiple slots identical to the slots 306A and 306B around the perimeter of the anchor 300 that create multiple deformable members.

It should also be appreciated that the slot and deformable member shapes illustrated in FIGS. 2 and 3 are intended merely as examples. In other examples, similarly suitable slots shapes may be cut from the provided bone anchor to create a deformable member that includes a zone of weakness having a width narrower than the deformable member's first and second portions. Additionally or alternatively, in some instances, the provided bone anchor may be heat treated at the respective zones of weakness such that the material is weakened at the zones of weakness. The material weakening causes the deformable members to deform at the respective zones of weakness, rather than at another location, when the bone anchor is transitioned to the expanded configuration. In such instances, the deformable members may or may not have a narrower width at the respective zones of weakness.

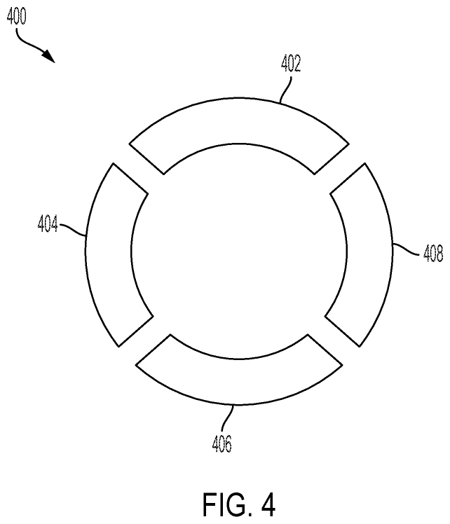

FIG. 4 illustrates a cross-sectional view of a middle portion (e.g., at the zones of weakness) of an example bone anchor 400 having four deformable members. The bone anchor 400 includes the deformable members 402, 404, 406, and 408 that are each separated by a slot. As stated above, the slots are an absence of material. The slots may be shaped according to the example slots described above.

As illustrated in FIGS. 2 and 3, a deformable member of the presently disclosed bone anchor may be individually symmetrical such that it includes a centered zone of weakness with first and second portions having equal lengths. The location of the zone of weakness of a respective deformable member contributes in part to the pull-out strength provided by the respective deformable member. For example, the location of the zone of weakness determines how the deformable member deforms when it expands outward, and thus determines a length of the deformable member that contacts bone, which contributes to the force that the deformable member exerts on the bone.

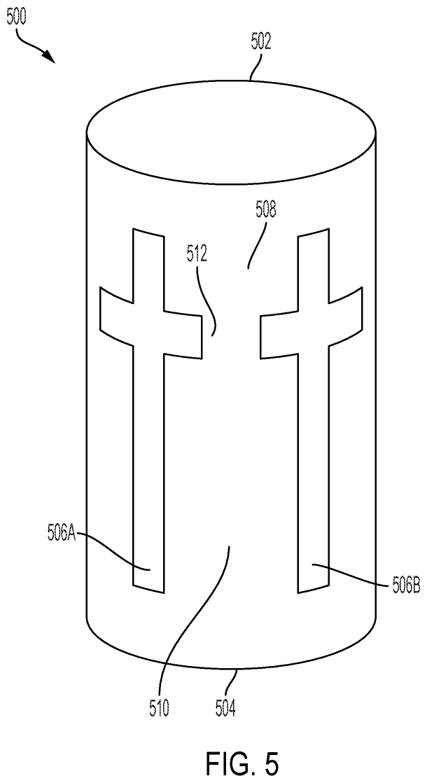

FIG. 5 illustrates an example bone anchor 500 having deformable members that are individually asymmetric. The bone anchor 500 includes a plunger end 502 and an insertion end 504. Additionally, the bone anchor 500 includes multiple cut-out slots, such as the slots 506A and 506B. The slots 506A and 506B are an absence of material. Between the slot 506A and the slot 506B is a deformable member that includes a zone of weakness 512 between a first portion 508 and a second portion 510. The deformable member is asymmetric in this example because the first portion 508 is shorter than the second portion 510. Stated differently, the zone of weakness 512 is positioned closer to the plunger end 502 than the insertion end 504. The bone anchor 500 therefore has a different expanded configuration as compared to the bone anchor 108 having individually symmetric deformable members in FIG. 1B.

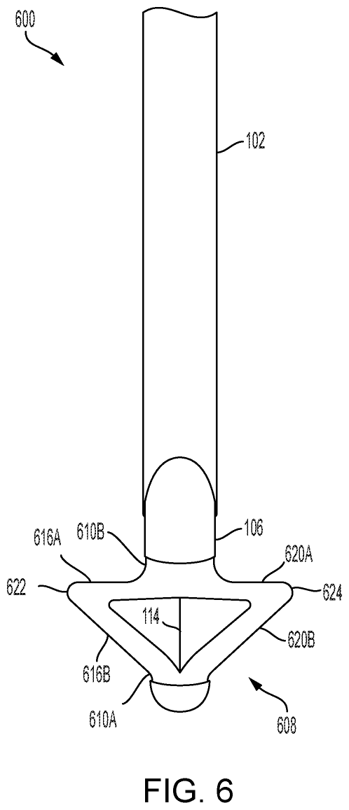

For instance, FIG. 6 illustrates a side view of an example bone anchor insertion system 600 having an example bone anchor 608 with an insertion end 610A and a plunger end 610B. The bone anchor 608 includes two deformable members that each have a first portion that is shorter than a second portion. The first deformable member includes a zone of weakness 622 between a first portion 616A and a second portion 616B. The second deformable member includes a zone of weakness 624 between a first portion 620A and a second portion 620B. As illustrated, because the first portion 616A and the first portion 620A are shorter than the second portion 616B and the second portion 620B, respectively, the first portion 616A and the first portion 620A are closer to the plunger end 610B when the bone anchor 608 is in an expanded configuration, as compared to the bone anchor 108 (FIG. 1B). In some instances, having the respective first portions 616A and 620A closer to the plunger end 610B may provide additional pull-out strength. Additionally, less of the bone anchor 608 resides within the bone hole when the respective first portions 616A and 620A contact the bone.

In some aspects of the present disclosure, the provided bone anchor's deformable members as a whole may be symmetrical such that they all expand outward symmetrically (e.g., FIGS. 1B and 6). In such aspects, the bone anchor exerts symmetric force on the bone across each of the deformable members. The symmetric force may be advantageous if the bone anchor is utilized with a flat bone surface. Many bone surfaces, however, are not flat and thus it may be advantageous in some instances to construct the bone anchor with asymmetric deformable members as a whole. A bone anchor having asymmetric deformable members as a whole exerts asymmetric force on the bone. Accordingly, a bone anchor having asymmetric deformable members as a whole may better conform to an uneven surface as compared to a bone anchor having symmetric deformable members as a whole.

In one example, the respective zones of weakness may have different locations on the deformable members on a bone anchor. This example results in a bone anchor having deformable members that expand outwardly different amounts and at different distances from the plunger end. In another example, the deformable members may be distributed unevenly around the bone anchor' perimeter. Some of the deformable members may be wider than other deformable members in such examples. For instance, the slots may be cut out of the bone anchor material in an asymmetric manner around the bone anchor's perimeter. This example results in a bone anchor having deformable members that are distributed asymmetrically around the bone anchor's perimeter. A bone anchor may also be constructed including both the above-described examples.

Accordingly, the bone anchor may be manufactured with a variety of shapes by varying the bone anchor width or length, the number of deformable members, the symmetry of an individual deformable member, and/or the symmetry of the deformable members as a whole. There are no limits on manufacturing larger bone anchors as described herein. Smaller versions of the bone anchors described herein are only limited by manufacturing techniques for cutting the slots and the size and number of the sutures required for a procedure. This flexibility enables a bone anchor with a wide variety of shapes, sizes, and/or pull-out strengths, which is not enabled by typical bone anchors.

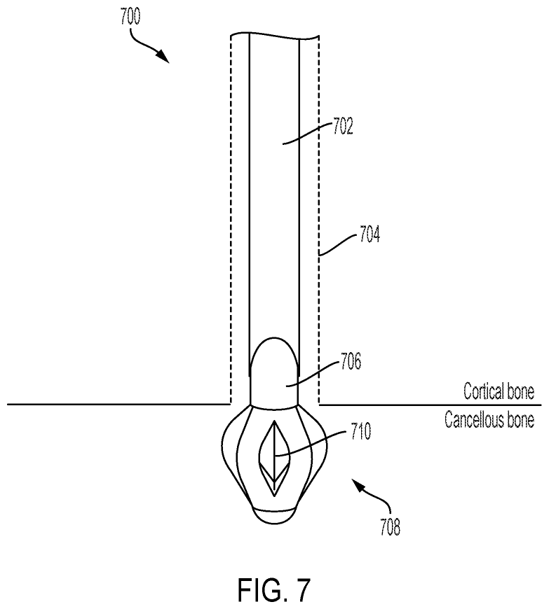

FIG. 7 illustrates a portion of an example method 700 of inserting a bone anchor 708 using the provided bone anchor insertion system. The bone anchor 708 is initially in a compact configuration within the tube 702. The tube 702 of the system may be installed into a bone hole 704. It should be appreciated that the bone hole 704 is illustrated much larger than the tube 702 for illustrative purposes only and the bone hole 704 more closely conforms to the tube 702 in practice. In an example, the tube 702 may be tapped into place in the bone hole 704 with a mallet until a marked depth is read on the exterior of the tube 702. This marked depth may correspond to a location that the surgeon prefers to implant the bone anchor 708, and thus the depth to which the tube 702 should be installed to position the bone anchor 708 at that location. For example, the surgeon may prefer to implant the bone anchor 708: (1) completely underneath the cancellous bone, (2) partly within the cancellous bone and partly underneath the cancellous bone, (3) completely within the cancellous bone, (4) partly within the cancellous bone and partly within the cortical bone, or (5) completely within the cortical bone. FIG. 7 illustrates the bone anchor 708 being implanted just below the cortical bone layer.

Once the tube 702 is properly positioned, in some instances, the tube 702 may then be retracted while the plunger 706 is maintained in position. In other instances, the plunger 706 may be depressed while the tube 702 is maintained in position. In either instance, the bone anchor 708 is thereby deployed from within the interior of the tube 702 while the plunger 706 remains in contact with the bone anchor 708 (e.g., the plunger end of the bone anchor 708). After the bone anchor 708 is deployed, the bone anchor 708 is transitioned to an expanded configuration. In some aspects, transitioning the bone anchor 708 to an expanded configuration involves tensioning at least one suture 710 while the plunger 706 is maintained in position to apply opposing force to the bone anchor 708. The at least one suture 710 may be tensioned manually, remotely by a robotic arm, or by another suitable mechanism. FIG. 7 illustrates the at least one suture 710 tensioned such that the bone anchor 708 has transitioned to an expanded configuration, though not a final expanded configuration.

As described above, in aspects in which the bone anchor 708 is constructed of a shape memory material with a pre-set shape, the bone anchor 708 transitions to an expanded configuration upon being deployed from the tube 702. In some instances, however, the pre-set shape that the shape memory material bone anchor 708 transitions to is not the preferred final expanded configuration based on the bone and/or ligament anatomy. For example, the pre-set shape may not suitably conform to the bone and therefore may provide less than desired pull-out strength.

Accordingly, whether or not the bone anchor 708 is constructed of a shape memory material, the at least one suture 710 may be tensioned to adjust the bone anchor 708 until a final expanded configuration is obtained. For example, a final expanded configuration may include the respective first portions of the deformable members of the bone anchor 708 contacting the cortical bone. Once the final expanded configuration is obtained, the at least one suture 710 may be clipped under arthroscopic visualization. The tension in the at least one suture 710 helps to stabilize the bone anchor 708 within the bone. The tube 702 and/or the plunger 706 may be removed from the bone hole 704 before or after the at least one suture 710 is clipped.

In some instances, after deploying the bone anchor 708, a surgeon may be dissatisfied with the expected results of transitioning the bone anchor 708 to an expanded configuration. In such instances, the surgeon may remove the provided bone insertion system without transitioning the bone anchor 708 to an expanded state. For example, the surgeon can remove the components without applying force to the bone anchor 708 with the plunger 706 so that the bone anchor 708 is not transitioned to an expanded configuration.

In another example, after transitioning the bone anchor 708 to an expanded configuration, a surgeon may be dissatisfied with the bone anchor 708 positioning, estimated pull-out strength, etc. In such an example, the surgeon may transition the bone anchor 708 back to a compact configuration by tensioning the at least one suture to pull the expanded bone anchor 708 back into the tube 702. The plunger 706 is translated away from the bone anchor 708 if needed so that it does not contact the bone anchor 708 as it is pulled back into the tube 702. The surgeon may then remove the bone anchor insertion system from the bone hole 704.

Without further elaboration, it is believed that one skilled in the art can use the preceding description to utilize the claimed inventions to their fullest extent. The examples and aspects disclosed herein are to be construed as merely illustrative and not a limitation of the scope of the present disclosure in any way. It will be apparent to those having skill in the art that changes may be made to the details of the above-described examples without departing from the underlying principles discussed. In other words, various modifications and improvements of the examples specifically disclosed in the description above are within the scope of the appended claims. For instance, any suitable combination of features of the various examples described is contemplated.

The invention is claimed as follows:

1. A bone anchor insertion system comprising:
an inserter including a tube;
a plunger to be depressed through an interior of the tube;
a bone anchor including an insertion end and a plunger end, the plunger end connected to the insertion end by a plurality of deformable members, each respective deformable member of the plurality of deformable members including a first portion, a second portion, and a third portion, the third portion between the first portion and the second portion, the third portion causing the respective deformable member to deform,
wherein the respective first portions are substantially parallel to the respective second portions when the bone anchor is in a compact configuration,
wherein the respective first portions are at an angle to the respective second portions when the bone anchor is in an expanded configuration,
wherein the bone anchor is positioned within the interior of the tube when the bone anchor is in the compact configuration, and
wherein forcing the insertion end towards the plunger end of the bone anchor in the compact configuration causes the bone anchor to transition to the expanded configuration; and
at least one suture coupled to the insertion end of the bone anchor.

2. The bone anchor insertion system of claim 1, wherein the tube includes a pointed tip to facilitate insertion of the inserter into a bone hole.

3. The bone anchor insertion system of claim 1, wherein the plunger is hollow and the at least one suture is positioned through the plunger.

4. The bone anchor insertion system of claim 1, wherein the bone anchor transitioning to the expanded configuration includes deforming the third portion.

5. The bone anchor insertion system of claim 1, wherein the respective third portions have a more narrow width than the respective first and second portions.

6. The bone anchor insertion system of claim 5, wherein the respective deformable members are tapered.

7. The bone anchor insertion system of claim 1, wherein the bone anchor in the compact configuration is cylindrical.

8. A bone anchor insertion system comprising:
an inserter including a tube;
a plunger to be depressed through an interior of the tube;
a bone anchor including an insertion end and a plunger end, the insertion end connected to the plunger end by a plurality of deformable members, each respective deformable member including a first portion and a second portion,
wherein the bone anchor is constructed of a shape memory material,
wherein the respective first portions are substantially parallel with the respective second portions when the bone anchor is in a compact configuration,
wherein the respective first portions are at an angle to the respective second portions when the bone anchor is in an expanded configuration,
wherein the bone anchor is positioned within the interior of the tube when the bone anchor is in the compact configuration, and
wherein the bone anchor transitions from the compact configuration to the expanded configuration upon the bone anchor exiting the interior of the tube; and
at least one suture coupled to the insertion end of the bone anchor.

9. The bone anchor insertion system of claim 8, wherein the shape memory material is nitinol.

10. The bone anchor insertion system of claim 8, wherein each of the respective deformable members are symmetric with one another.

11. The bone anchor insertion system of claim 8, wherein the respective deformable members are asymmetric.

12. The bone anchor insertion system of claim 8, wherein the plurality of deformable members is equal to three or four deformable members.

13. The bone anchor insertion system of claim 8, wherein the respective first portions and the respective second portions have equal lengths.

14. The bone anchor insertion system of claim 8, wherein the respective first portions and the respective second portions have unequal lengths.

15. The bone anchor insertion system of claim 8, wherein the bone anchor in the compact configuration has a first width and the bone anchor in the expanded configuration has a second width, and the second width is less than or equal to four times the first width.

16. A bone anchor insertion method comprising:
inserting a bone anchor coupled to an inserter through a bone, the inserter including a tube and a plunger to be depressed through an interior of the tube, the bone anchor including an insertion end connected to a plunger end by a plurality of deformable members, each respective deformable member including a first portion and a second portion, wherein the bone anchor is initially in a compact configuration within the interior of the tube, the respective first portions being substantially parallel with the respective second portions when the bone anchor is in the compact configuration, and wherein at least one suture including a coupling end and a free end is coupled to the insertion end of the bone anchor at the coupling end;
retracting the tube while maintaining a positioning of the plunger, thereby causing the bone anchor to exit from the interior of the tube;
transitioning the bone anchor from the compact configuration to an expanded configuration, wherein the respective first portions are at an angle to the respective second portions when the bone anchor is in an expanded configuration; and removing the inserter from the bone.

17. The bone anchor insertion method of claim 16, wherein each respective deformable member includes a zone of weakness between the respective first and second portions, and wherein the bone anchor is transitioned from the compact configuration to the expanded configuration by pulling the free end of the at least one suture while the plunger end of the bone anchor remains in contact with the plunger.

18. The bone anchor insertion method of claim 16, wherein the bone anchor is constructed of a shape memory material, and wherein the bone anchor transitions from the compact configuration to the expanded configuration after exiting the interior of the tube based on a pre-set shape of the shape memory material.

19. The bone anchor insertion method of claim 18, further comprising pulling the free end of the at least one suture while the plunger end of the bone anchor remains in contact with the plunger.

20. The bone anchor insertion method of claim 16, wherein the plunger is depressed when the bone anchor is inserted entirely past cortical bone.
